(19) United States
(12) Patent Application Publication
(10) Pub. No.: US 2013/0144153 A1
(43) Pub. Date: Jun. 6, 2013

(54) FUNCTIONAL MAGNETIC RESONANCE IMAGING APPARATUS AND METHODS

(71) Applicant: The Regents of the University of California, Oakland, CA (US)

(72) Inventors: Benjamin Inglis, Lafayette, CA (US); Michael Moessle, Bonn (DE); Michael Hatridge, New Haven, CT (US); Sarah Busch, Washington, DC (US); Steven Conolly, Palo Alto, CA (US); John Clarke, Berkeley, CA (US); Paul Sangiorio, Oakland, CA (US)

(73) Assignee: The Regents of the University of California, Oakland, CA (US)

(21) Appl. No.: 13/689,434

(22) Filed: Nov. 29, 2012

Related U.S. Application Data
(60) Provisional application No. 61/565,629, filed on Dec. 1, 2011.

(51) Int. Cl. A61B 5/055

(52) U.S. Cl. CPC A61B 5/055
USPC 600/409

(57) ABSTRACT

This disclosure provides systems, methods, and apparatus related to functional magnetic resonance imaging. In one aspect, a method may include generating a bulk magnetization in a subject, establishing a longitudinal relaxation time constant (T₁) contrast between constituents in the subject, creating spatially localized magnetic resonance (MR) signals from the subject, detecting the MR signals, and generating a functional image of the subject.

### RELATED APPLICATIONS

This application claims priority to U.S. Provisional Patent Application No. 61/565,629, filed Dec. 1, 2011, which is herein incorporated by reference.

### STATEMENT OF GOVERNMENT SUPPORT

This invention was made with government support under Contract No. DE-AC02-05CH11231 awarded by the U.S. Department of Energy. The government has certain rights in this invention.

### FIELD

Embodiments disclosed herein relate to the field of magnetic resonance imaging (MRI), and particularly relate to a method of performing functional brain imaging of brain tissue in a living human via magnetic resonance imaging (MRI).

### BACKGROUND

Functional magnetic resonance imaging (fMRI) has revolutionized neuroscience in the past twenty years. FMRI is now being used to investigate such phenomena as aging, brain plasticity during recovery from injury (e.g., post-stroke), learning and development, lie detection, pain management, and behavior (e.g., economic behavior). Since its first demonstration in humans in 1992, there have been over 250,000 published reports. It is also being used for neurosurgical planning for tumor resection and a Current Procedural Terminology (CPT) Code has been issued by the American Medical Association for that application; a CPT code is a necessary step for insurance reimbursements. CPT codes for other applications of fMRI seem likely to follow. Brain imaging methods, and fMRI in particular, are poised to become widespread tools in all aspects of behavioral and neurological medicine.

### SUMMARY

The systems, methods, and devices of the disclosure each have several innovative aspects, no single one of which is solely responsible for the desirable attributes disclosed herein.

Some embodiments disclosed herein provide a method of performing functional brain imaging of brain tissue in the brain of a living human via magnetic resonance imaging (MRI), resulting in at least one functional brain image. In some embodiments, the method includes generating a bulk magnetization of the head, establishing a longitudinal relaxation time constant (T₁) contrast between constituents in the brain, creating spatially localized magnetic resonance (MR) signals from the brain tissue, detecting the MR signals, and generating the functional brain image.

Another innovative aspect of the subject matter described in this disclosure can be implemented in a method including generating a bulk magnetization in a subject, establishing a longitudinal relaxation time constant (T₁) contrast between constituents in the subject, creating spatially localized magnetic resonance (MR) signals from the subject, detecting the MR signals, and generating a functional image of the subject.

Another innovative aspect of the subject matter described herein can be implemented in an apparatus including a pre-polarizing magnet, a secondary magnet, gradient electromagnetic coils, rotating magnetic field transmission coils, a detection mechanism, and a controller. The detection mechanism is selected from the group consisting of one or more magnetometers and one or more inductive pickup coils. For example, the detection mechanism may be selected from the group consisting of one or more SQUIDs, a MEG helmet, one or more copper coils, and one or more superconducting alloy coils. The controller includes program instructions for conducting a process including the operations of: generating a bulk magnetization in a subject using the pre-polarizing magnet; establishing a longitudinal relaxation time constant (T₁) contrast between constituents in the subject using a sequence of constant and time-varying magnetic fields; creating spatially localized magnetic resonance (MR) signals from the subject using an imaging pulse sequence; detecting the MR signals using the detection mechanism; and generating a functional image of the subject.

### BRIEF DESCRIPTION OF THE DRAWINGS

FIG. 1 shows an example of the vascular space occupancy (VASO) method.

FIG. 2 shows an example of a method of performing fMRI.

FIG. 3 shows an example of a timing diagram according to embodiments disclosed herein.

FIG. 4 shows an example time course of signal measurements from an image region at the repetition time, TR, while the subject performs a mental task.

FIG. 5 shows an example of a cross-sectional schematic illustration of a device utilizing inductive signal detection (i.e., an inductively detected pre-polarized fMRI (ip-fMRI) device).

FIG. 6 shows an example of a cross-sectional schematic illustration of a magnetometer-detected fMRI device (i.e., a magnetometer-detected pre-polarized fMRI (mp-fMRI) device).

### DETAILED DESCRIPTION

### Introduction

FMRI is currently performed at constant field strengths, typically 1.5 tesla (T) and higher. FMRI measurements performed at constant magnetic fields are referred to as ‘high field’ measurements. In high field fMRI, functional contrast is most often achieved via magnetic susceptibility changes of blood pools, commonly referred to as Blood Oxygenation Level-Dependent (BOLD) contrast. Alternative strategies may involve contrast based on changes of cerebral blood flow (CBF) or cerebral blood volume (CBV).

The common strategy to all fMRI methods, however, is that neural activation leads to increased metabolic demand and so to changes in oxygen extraction fraction (OEF), CBF, and CBV. These changes peak about 4 to 6 seconds after stimulus onset. Images with the contrast mechanism of choice are acquired in a time series, during which the subject (e.g., a human or an animal) performs tasks, is provided with sensory stimuli, or is monitored during the so-called “resting state” condition. The time series of images is then taken off-line for statistical evaluation of signal changes.

However, fMRI as currently performed utilizes high-field superconducting magnets at field strengths of 1.5 T and higher. In fact, higher magnetic fields are widely accepted to be preferable for fMRI. Higher fields do have many significant advantages, but one significant drawback is the high cost of the equipment and the complexity of siting. For example, a 3 T scanner costs around $2.5 million, and siting typically costs a further $1 million for special foundations and stray magnetic field shielding. The superconducting magnet requires liquid helium fills every six months, resulting in a cost overhead of at least $50,000 per year. For fMRI to become as widely practiced as its potential suggests it could be, a methodology is required to reduce the unit cost and to allow installation of fMRI scanners into smaller spaces.

Many potential subjects for fMRI may have contraindications, such as deep brain stimulators, metal skull plates, and other implanted devices that are incompatible with high field (1.5 T and higher) scanners for either safety or imaging performance reasons. For example, a patient who has had part of his skull resected to alleviate cranial pressure following a traumatic injury could be a candidate for fMRI, yet the regions of brain immediately underneath the surgical intervention, which would likely be the most critical anatomical structures to investigate, could be rendered invisible to conventional high field MRI because of strong magnetic susceptibility gradients arising from the biomaterials.

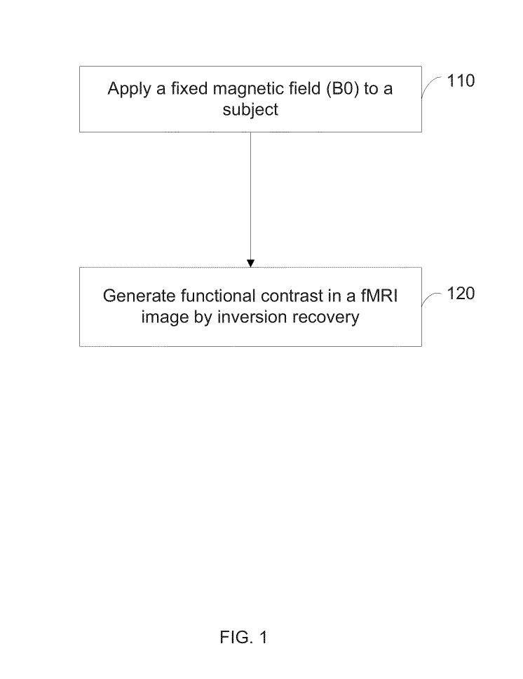

U.S. patent application Ser. No. 10/525,699, filed Aug. 26, 2003, is directed to the original vascular space occupancy (VASO) method¹³, also described in a publication⁶. In the original VASO method, the magnetic field strength (B₀) was fixed during the experiment, and functional contrast was generated by inversion recovery (i.e., an inversion B, pulse and recovery delay during B₀), as shown in FIG. 1.

U.S. Pat. No. 7,071,689 is directed to a method to obtain multi-slice VASO images using a constant field MRI system¹⁴. U.S. Pat. No. 6,462,544 is directed to one-sided pre-polarized MRI systems¹⁵. The intended use of such systems is to allow open-access scanning (e.g., for claustrophobic subjects, or for surgical access). One-sided PMRI systems tend to generate weaker polarizing fields and may not have the performance characteristics necessary to perform fMRI.

Magnetoencephalography (MEG) is another brain imaging method which is gaining popularity (e.g., for pre-surgical planning) MEG scanners utilize arrays of hundreds of superconducting quantum interference device (SQUID) magnetometers to detect the magnetic fields from the brain. The biggest limitation of this method is that localization accuracy is fundamentally limited by the ‘inverse problem’—the absence of a unique solution to solve for the magnetic fields inside the brain.

Some embodiments disclosed herein provide a low-field magnetic resonance imaging (MRI) method for the measurement of functional activation in subjects (e.g., human brains). The functional contrast arises from changes in cerebral blood volume (CBV), which is typically on the order of about 2% to 5% in grey matter at rest, and increases to 3% to 8% upon neural stimulation. The CBV increase could be measured as a signal change—typically reduction of gray matter signal—by generating T₁ relaxation contrast with field cycling of one or more pre-polarizing field strengths and one or more evolution field strengths, in accordance with some embodiments disclosed herein. T₁ is the longitudinal relaxation time constant. The resultant contrasted MR signal is detected using conventional MRI pulse sequences.

Some embodiments disclosed herein could improve or optimize CBV contrast by taking into account the large differences between blood and gray matter T₁ values that emerge at low magnetic fields. Thus, the lower signal-to-noise ratio (SNR) produced at low magnetic fields could be offset by the higher intrinsic contrast level—and hence attain acceptable contrast-to-noise ratio (CNR)—leading to a useful fMRI method.

Some embodiments disclosed herein include (1) using pre-polarizing magnetic fields to attain SNR sufficient for MR imaging, (2) field cycling to achieve T₁ contrast, and (3) functional brain imaging contrast based on CBV changes (i.e., the vascular space occupancy (VASO) method). Thus, some embodiments include a pre-polarized VASO (p-VASO) method.

Some embodiments disclosed herein modify B₀ as well as one or more evolution (Bₑ) and pre-polarization (Bₚ) fields, in combination with an inversion B₁ pulse, to obtain the T₁ contrast. A device using embodiments disclosed herein could resemble more a traditional ‘donut’ or tube-based MRI scanner where the human subject is inserted into the magnet bore.

### Method

Some embodiments disclosed herein provide a method of performing functional brain imaging of brain tissue via magnetic resonance imaging (MRI). In some embodiments, the method includes (1) generating a bulk magnetization in a subject (e.g., the brain of a human being), (2) establishing a longitudinal relaxation time constant (T₁) contrast between constituents in the brain tissue, (3) creating spatially localized magnetic resonance (MR) signals from the brain tissue, (4) detecting the MR signals, and (5) generating a functional brain image.

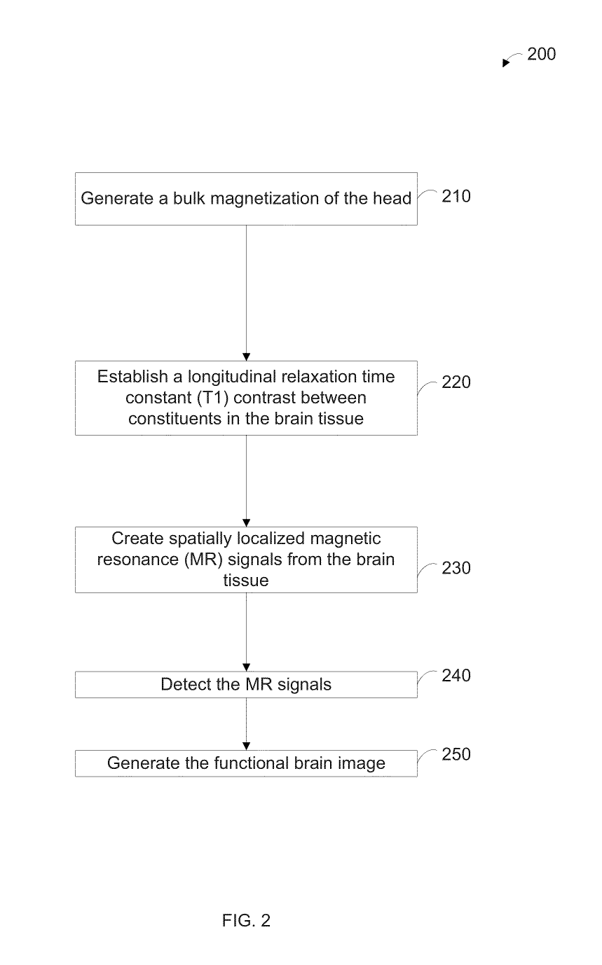

FIG. 2 shows an example of a method of performing fMRI. The method 200 begins with operation 210 of generating a bulk magnetization in the head of a human subject. In operation 220, a longitudinal relaxation time constant (T₁) contrast between constituents in the brain tissue is established. In operation 230, spatially localized magnetic resonance (MR) signals from the brain tissue are created. In operation 240, the MR signals are detected. In operation 250, a functional image of the brain is generated.

In some embodiments, operation 210 includes applying at least one initial polarizing magnetic field (Bₚ) pulse to the brain.

In some embodiments, operation 220 includes applying a magnetic field-cycled pulse sequence to the brain tissue. In some embodiments, the magnetic field-cycled pulse sequence may include at least one constant magnetic field pulse (B₀), at least one oscillating magnetic field pulse (B₁), at least one evolution magnetic field pulse (Bₑ) or at least one additional polarizing magnetic field pulse (Bₚ).

In some embodiments, operation 220 includes placing blood magnetization of the brain tissue at a null value at the time of operation 240. In some embodiments, operation 220 includes placing blood magnetization of the brain tissue near a null value at the time of operation 240.

In some embodiments, operation 230 includes applying at least one readout oscillating and gradient magnetic field pulse sequence to the brain tissue.

In some embodiments, operation 240 includes detecting the MR signals via magnetic induction. In some embodiments, operation 240 includes detecting the MR signals via magnetic induction with at least one inductive pickup coil. In some embodiments, operation 240 includes detecting the MR signals via magnetic induction with at least one magnetometer.

In some embodiments, the MR signals may be detected via magnetic induction with at least one SQUID. In some embodiments, the MR signals may be detected via magnetic induction with a plurality of SQUIDS. In some embodiments, the localization of magnetoencephalography (MEG) signals may be obtained along with the detected MR signals.

In some embodiments, operation 250 includes measuring functional activation in the brain tissue from the detected MR signals. In some embodiments, operation 250 includes outputting a representation of the measured functional activation.

In some embodiments, operation 250 includes measuring functional deactivation in the brain tissue from the detected MR signals. In some embodiments, operation 250 includes outputting a representation of the measured functional deactivation.

### EXAMPLE

Embodiments disclosed herein will be described in greater detail by way of a specific example. The following example is offered for illustrative purposes, and is intended not to limit the embodiments in any manner.

Embodiments disclosed herein use field cycling. T₁ contrast utilizing field cycling has been demonstrated for other applications²⁻⁵. In the embodiments disclosed herein, T₁ contrast achieved with field cycling is used to emulate the high field method called Vascular Space Occupancy (VASO), which uses the intrinsic (fixed) T₁ differences between blood and gray matter to measure human brain function on constant-field MRI systems⁶. Some embodiments disclosed herein use field cycling to exploit the large differences in T₁ between blood and brain that exist at fields below 500 mT. At magnetic fields above 1.5 T, the differences between blood and gray matter T₁ are typically on the order of 10% to 40%, whereas the differences can be a factor of two or more at very low fields. This allows some of the embodiments disclosed herein to offset the lower SNR of pre-polarized MRI with the higher contrast available at low fields. Some embodiments disclosed herein could produce functional contrast-to-noise ratios to rival 1.5 T fMRI.

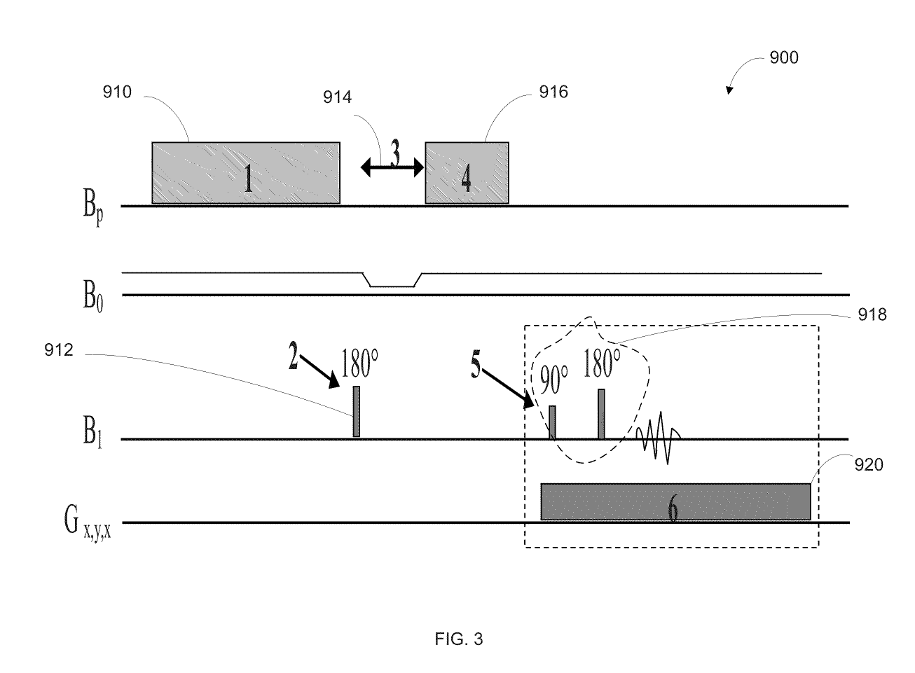

FIG. 3 shows an example of a timing diagram according to some embodiments disclosed herein. As shown in FIG. 3, the events in the timing diagram 900 includes:
(1) a pulsed pre-polarization magnetic field, Bₚ, of about 400 mT (pre-polarizing pulse) in a first time period 910;
(2) an inversion B₁ pulse in a second time period 912;
(3) an evolution period under a low magnetic field Bₑ, (low field evolution period) in a third time period 914;
(4) a second period of about 400 mT magnetic field (second pre-polarizing pulse) in a fourth time period 916;
(5) generation of detectable magnetization during the readout field B₀ in a fifth time period 918; and
(6) image formation and signal detection in a sixth time period 920.
   The events in time periods 910, 912, 914, and 916 involve cycling of the applied static magnetic fields, Bₚ, Bₑ and B₀, and lead to a T₁ contrast between blood and brain tissue.

The effect of this field cycling contrast module is to place the blood magnetization at or near a null at the time of signal detection. Some embodiments disclosed herein are a field-cycled variant of the VASO method. Contrast may be increased or maximized by taking advantage of the large inherent difference between T₁s for blood and brain tissue at the low magnetic fields. Some embodiments disclosed herein could utilize maximum T₁ contrast between blood and brain tissue instead of a null⁷, or could even be set to the inverse contrast mechanism where brain tissue signals are set low and the blood signal is preferentially detected.

B₁ pulse frequencies in time periods 912, 918, and 920 may be determined by the static magnetic field present at the time of their application (i.e., the appropriate Larmor frequencies). In this example, three different magnetic fields were used, but two or more fields could be used by embodiments disclosed herein. Image formation and signal detection could follow any of a number of standard strategies common to conventional MRI. Magnetic resonance signals are detected by induction, with a single coil or a coil array such as that for parallel imaging (in order to accelerate the spatial encoding), with a SQUID or SQUID array, or with other magnetometers. Note that the events and time periods shown in FIG. 3 could be used by inductively detected pre-polarized fMRI (ip-fMRI), magnetometer-detected pre-polarized fMRI (mp-fMRI), and MEG-fMRI devices.

Functional brain imaging examinations using embodiments disclosed herein could be conducted in a manner similar to high field functional MRI. One difference is that functional contrast would be based on T₁ differences between blood and brain tissue that could be exploited at low fields by using field cycling preparation before signal detection in accordance with some embodiments. Embodiments disclosed herein map human brain function in a series of steps. First, the embodiments disclosed herein measure the T₁ contrast established by field cycling using any one of a number of previously described imaging or spectroscopic pulse sequences. In some embodiments, a conventional fast imaging sequence such as Fast Spin Echo or Echo Planar Imaging may be used. Embodiments disclosed herein next acquire the field-cycled, T₁ contrasted signals (e.g., images) in regularly spaced intervals, typically about 2 seconds apart. During a time series of these acquisitions, the human subject is asked to perform tasks or is provided with stimuli such as pictures or sounds, or may be studied in the absence of any particular task in the so-called “resting state.” These stimuli or tasks generate activity away from baseline in specific portions of the human brain, leading to CBV changes in the tissue encompassing the activated or deactivated neurons. Because of the different T₁ values for blood and tissue, a change of CBV implies a commensurate change of signal level. Note that the specific timing of the sequence or the signal detected—either blood as an increased or decreased signal, or tissue as a decreased or increased signal—is not limited, though in some embodiments, the tissue signal would be detected because it is larger.

Further, functional MRI according to embodiments disclosed herein could follow the approach for fMRI at high magnetic fields (typically 1.5 T and above) using contrast mechanisms such as BOLD, (constant field) VASO, and arterial spin labeling (ASL). Yet further, the embodiments disclosed herein could be used by the ip-fMRI, the mp-fMRI, and the MEG-fMRI devices. Again, in some embodiments, a time series of spatially localized measurements, either images or spectra, may be acquired from a subject's brain according to an event sequence as shown in FIG. 3 for functional contrast, while the subject performs some sort of mental task. The time series of measurements is then processed with some kind of activity model, the most common being cross-correlation with an assumed task time course. The particular task, the duration of the time series, the processing model and other experimental variables is not limited by the contrast mechanism and could involve a resting period to study ongoing mental activity, or any task-based paradigm (e.g., the presentation of visual cues to execute a particular instruction), as with using BOLD at high magnetic fields.

Illustration of an fMRI experiment utilizing embodiments disclosed herein may be constructed by analogy to task-based fMRI with BOLD contrast at high field. Recognizing that the vascular changes following neural activation or deactivation, which cause alterations in metabolic rate, occur over a time scale of 4 to 6 seconds, a minimum sampling rate—the so-called Nyquist condition—of approximately two seconds per measurement is implied, although the particular measurement rate is limited only by the timing required to achieve field-cycled T₁ contrast, as shown in FIG. 3.

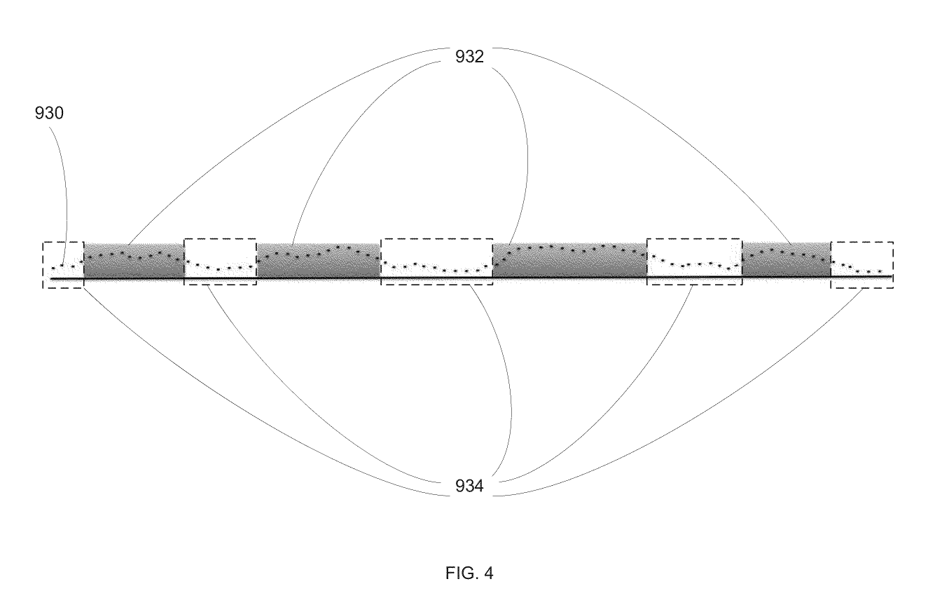

The measurement is then repeated at the repetition time, TR, while the subject performs a mental task, as shown in FIG. 4, where an example time course of signal measurements from an image region has been simulated by a dotted line 930. Dotted line 930 is the time-dependent signal level in that image region. Manipulation of the subject's mental state is represented by boxes 932, while boxes 934 represent a baseline, or control, condition. Cross-correlation of the dotted line 930 (i.e., the signal time course) with the task time course represented by the boxes 932 produces a statistical map that indicates the strength of brain activity resulting from the task. As with conventional fMRI, the duration of the task and control periods, the sampling rate (TR), the number of task/control periods and other aspects of the experimental paradigm may be changed.

Again, FIG. 4 is an illustration of an example task-based fMRI experiment utilizing embodiments disclosed herein. Measurements are obtained approximately every two seconds, as indicated by dotted line 930, using a process as shown in FIG. 3 during which the patient's mental state is manipulated. In this example, a task state is indicated by the boxes 932 while a control state is indicated by the boxes 934. Correlation of the task time course 932 with a region-of-interest from the contrasted data 930 obtained via embodiments disclosed herein (e.g., a pixel in a particular brain region) may determine the strength of mental activity being invoked in the patient's brain by the task.

### Apparatus

Another aspect of the implementations disclosed herein is an apparatus configured to accomplish the methods described herein. A suitable apparatus includes hardware for accomplishing the process operations and a system controller having instructions for controlling process operations in accordance with the disclosed implementations. Hardware for accomplishing the process operations includes fMRI apparatus, including ip-fMRI apparatus, mp-fMRI apparatus, and MEG-fMRI apparatus. The system controller will typically include one or more memory devices and one or more processors configured to execute the instructions so that the apparatus will perform a method in accordance with the disclosed implementations. Machine-readable media containing instructions for controlling process operations in accordance with the disclosed implementations may be coupled to the system controller.

As noted above, three broad classes of instruments could utilize embodiments disclosed herein. In a first class, inductively detected pre-polarized fMRI (ip-fMRI) devices, the polarizing and evolution magnetic fields could be produced with conventional (non-superconducting) electromagnets, making an ip-fMRI device cheaper and easier to site (smaller stray field and reduced mass) than conventional fMRI. An ip-fMRI device using embodiments disclosed herein would likely be an order of magnitude lower in cost than a conventional fMRI scanner

It is anticipated that a device utilizing the embodiments disclosed herein as the functional contrast mechanism could be produced for about one tenth of the cost of a conventional fMRI scanner and could be sited in standard office/lab spaces with only modest specialist requirements, such as an anti-vibration stand, conditioned electrical power and a chilled water supply. This device could use copper coils for detection of the MR signals. A head-sized device having similar specifications could be expected to be capable of reasonably using the embodiments disclosed herein.

In a second class, magnetometer-detected pre-polarized fMRI (mp-fMRI) devices, the signal level could be enhanced by pre-polarization while the MR signals could be detected by a superconducting quantum interference device (SQUID), or other magnetometer, instead of an inductive pickup coil (as for conventional fMRI and as with ip-fMRI). An mp-fMRI device could work at lower detection fields than an ip-fMRI device, leading to further performance gains in exchange for a more complicated detection system.

A device utilizing sensitive magnetometers, such as SQUIDs, and capable of performing mp-fMRI, according to embodiments disclosed herein, could negate the safety and/or performance limitations that higher magnetic fields present. While the temporal and spatial performance of mp-fMRI might not match the capabilities of a high field scanner for a normal patient, in the case of these certain patient sub-groups, the availability of an mp-fMRI scanner may be the only way to obtain fMRI results.

Signal detection via magnetometers such as SQUIDs could permit operation at lower readout magnetic field strength (B₀) than for the ip-fMRI variant. This would likely translate into a performance benefit, under certain circumstances, via the increased range of T₁ values attainable; greater T₁ contrast could feasibly be attained from the p-VASO scheme. However, SQUIDs and other magnetometers are more complicated and expensive to build than simple copper detection coils, as for ip-fMRI, and it is therefore envisaged that an instrument capable of mp-fMRI using the embodiments disclosed herein would be more expensive, albeit with higher potential contrast.

SQUID detection, however, also raises the possibility of a third class of instrument, a combination device that can measure magnetoencephalography (MEG) and fMRI in situ, an MEG-fMRI device, using the embodiments disclosed herein. The fMRI signals could then be used to constrain the localization of MEG signals, for example, greatly enhancing the spatial accuracy of the latter technique.

Embodiments disclosed herein provide for obtaining additional spatial information on the activity in a human brain (e.g., from concomitant fMRI changes), and thus for constraining the mathematical solutions of MEG, improving the spatial accuracy of the MEG method.

As noted above, SQUID magnetometers may be used to detect MR signals in place of the inductive pickup coils used for conventional MRI detection.¹ Embodiments disclosed herein could extend the utility of an instrument engineered for in situ MEG and MRI acquisitions. In essence, such a simultaneous device could allow the detection of the magnetic fields as well as the hemodynamic changes that accompany neural activity. Embodiments disclosed herein output signals that have an exact spatial solution that could be used to improve the MEG localization.

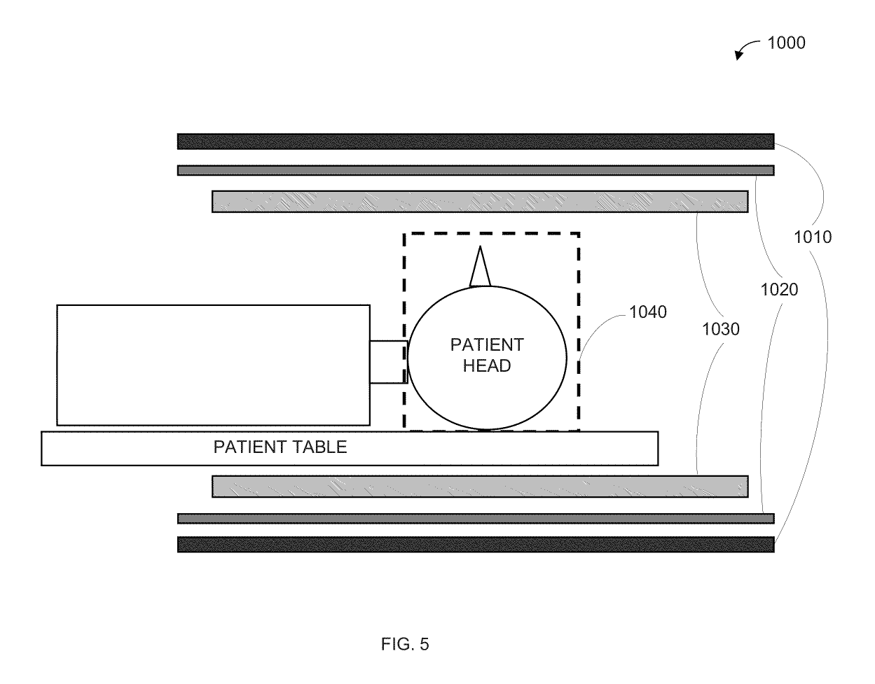

FIG. 5 shows an example of a cross-sectional schematic illustration of a device 1000 utilizing inductive signal detection (ip-fMRI). The device 1000 includes (1) a pre-polarizing magnet 1010 (pre-polarizing magnetic field coil, Bₚ), (2) a secondary magnet 1020 (evolution and readout magnetic field coil, Bₑ and B₀), (3) gradient electromagnetic coils 1030 for spatial encoding (X, Y, Z gradient coils), and (4) B₁ rotating magnetic field transmission and detection coils 1040 (B₁ coil system). The pre-polarizing magnet 1010 may be capable of attaining pulsed magnetic fields up to about 600 mT. The secondary magnet 1020 may be capable of attaining lower evolution/readout fields, one to two orders of magnitude lower than the pre-polarizing magnet 1010. The coil arrangement of the device 1000 uses inductive coil detection. In some embodiments, the magnetic field coils are tubular (e.g., solenoid-like).

The hardware elements of the device 1000 use existing, conventional technologies. One requirement for the device 1000 is that it be possible to cycle between higher and lower fields to attain sufficient SNR and to generate field-cycled T₁ contrast. The hardware could use existing designs, as being developed at the Conolly Lab, University of California, Berkeley, for pre-polarized MRI (PMRI) of human limbs⁸⁻¹¹. Embodiments disclosed herein utilize this type of hardware to take advantage of a particular physiological phenomenon—namely the CBV change following neural activation—and the associated T₁-weighted signal change.

In some embodiments, the electromagnets required for polarization, field cycling, and spatial encoding (the so-called gradient coils) would be head-sized (i.e., having a usable diameter of spherical volume of about 30 cm). The precise size and geometry of these coils is not limited, and is instead determined by specific considerations of detection pulse sequence choice and other standard MR criteria. For whole-brain imaging, for example, it would be important that all blood resident in the brain during the time of a field-cycled acquisition experiences approximately the same magnetic fields.

An ip-fMRI device according to embodiments disclosed herein could have a low cost (compared to conventional fMRI) and have relatively easy siting requirements. Additionally, the lower polarizing magnetic fields could render the device quieter than conventional fMRI. This could be advantageous for noise-susceptible populations and for acoustic fMRI studies where the high acoustic noise (greater than 90 dB) of conventional fMRI scanners can be a confounding factor. The lower magnetic fields could also render embodiments disclosed herein appropriate for functional scanning of patients having implants such as neurostimulators, aneurism clips, and other contraindications for high field fMRI. Finally, it has been demonstrated that contrast based on CBV changes has better spatial specificity than those from conventional (BOLD) contrast¹². Thus, while the contrast-to-noise ratio (CNR) may be marginally lower when using embodiments disclosed herein compared to BOLD contrast at 1.5 T, the fidelity of the signals being measured could allow a high degree of confidence.

Embodiments disclosed herein could result in lower SNR than conventional fMRI. Using field cycling, however, embodiments disclosed herein could attain CNR that is comparable to 1.5 T fMRI using BOLD contrast.

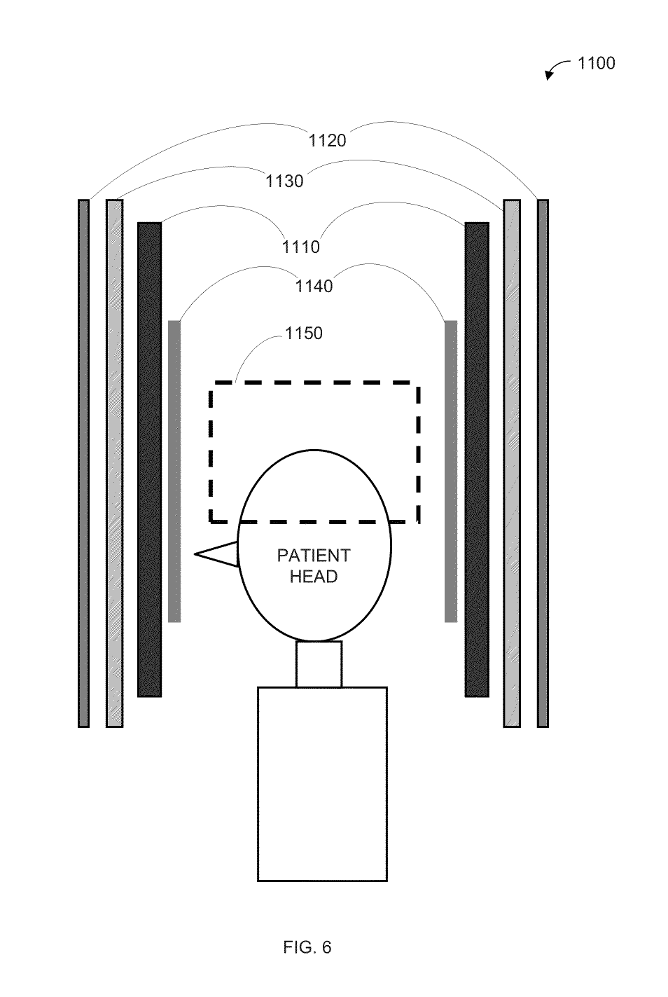

FIG. 6 shows an example of a cross-sectional schematic illustration of a magnetometer-detected fMRI device (mp-fMRI device). An mp-fMRI device may include a single SQUID detector, another magnetometer, or an array of detectors. As shown in FIG. 6, in some embodiments, a mp-fMRI device 1100 may include (1) a pre-polarizing magnet 1110 (pre-polarizing magnetic field coil, Bₚ), (2) a secondary magnet 1120 (evolution and readout magnetic field coil, B₀), (3) gradient electromagnetic coils 1130 for spatial encoding (X, Y, Z gradient coils), (4) a B₁ rotating magnetic field transmission coil 1140, and (5) a SQUID or other detector array coil system 1150 (e.g., an array of SQUIDs similar to a conventional MEG ‘helmet,’ as has been used for structural MRI¹⁷). The pre-polarizing magnet 1110 may be capable of attaining pulsed magnetic fields up to about 200 mT. The secondary magnet 1120 may be capable of attaining lower evolution/readout fields, typically three to four orders of magnitude lower than the pre-polarizing magnet 1110.

A mp-fMRI device according to the embodiments disclosed herein could be used for patients having implanted metal devices. Magnetometers such as SQUIDs could detect magnetic fields (e.g., at low frequencies) that are orders of magnitude smaller than can be detected with conventional induction in a copper coil. Thus, it could be feasible to operate the mp-fMRI scanner, using embodiments disclosed herein, at lower overall field strengths, both for pre-polarization and signal detection, providing additional safety to the patient (via reductions in the amount of force and current generated in a conducting implant) but without compromising the functional MRI performance. Indeed, it could possible that the contrast-to-noise ratio could be enhanced at the lower magnetic fields via access to a wider range of T₁ values.

A MEG-fMRI device could employ some of the components depicted in FIG. 6. The MEG and fMRI detection modes could be interleaved in accordance with some embodiments. Thus, a large array of SQUID detectors as used in modern MEG scanners could be used in the MEG-fMRI in addition to the components shown in FIG. 6. Such a MEG-fMRI device would have very different operating characteristics than the mp-fMRI device. When in MEG mode, the MEG-fMRI device would require very low background noise, 1 fT/√Hz or less. This would require low noise electronics and schemes to eliminate all sources of extraneous electromagnetic fields very quickly between modes.

Adoption of simultaneous MEG and fMRI in the form of the MEG-fMRI device could be driven by methodological considerations, in particular the enhanced fidelity of MEG signal localization provided by the in situ fMRI, versus the additional complexity and cost of hardware to enable embodiments disclosed herein. However, 6.8 million Euros funding over four years provided by the European Union to construct devices capable of simultaneous MEG with structural MRI at four universities¹⁶ suggests that there is sufficient academic interest to pursue these combined methodologies for the research market at least. One research group has constructed a combined device capable of performing structural MRI with the same SQUID array as can also be used for MEG¹⁸.

Simultaneous electroencephalography (EEG) with high field fMRI has garnered considerable interest in the past decade, because these methods are considered to be complimentary. FMRI could be used to provide high spatial resolution while EEG provides high temporal resolution. Similar benefits exist for simultaneous (interleaved) MEG-fMRI. MEG is usually considered to be a higher fidelity recording of electromagnetic brain activity than EEG because it does not suffer from current blurring across the scalp, as does EEG. However, EEG-MEG experiments have also been utilized, with each method providing slightly better discrimination of certain dipole field orientations. Thus, an EEG system could also be interfaced with the proposed MEG-fMRI device according to embodiments disclosed herein to provide even better fidelity, as well as a unique window into neurovascular coupling.

While the embodiments described herein are directed to performing functional brain imaging of brain tissue in a living human being, the methods are also applicable to fMRI performed on the brain tissue of other living organisms and fMRI performed on other organs/tissue (e.g., the spinal cord) in living organisms.

It is to be understood that the above description and examples are intended to be illustrative and not restrictive. Many embodiments will be apparent to those of skill in the art upon reading the above description and examples. The scope of the embodiments disclosed herein should, therefore, be determined not with reference to the above description and examples, but should instead be determined with reference to the appended claims, along with the full scope of equivalents to which such claims are entitled. The disclosures of all articles and references, including patent applications and publications, are incorporated herein by reference for all purposes.

### REFERENCES

1. J Clarke, R McDermott, A Pines, A H Trabesinger. SQUID detected NMR and MRI at ultralow fields. U.S. Pat. No. 7,053,610. May 30, 2006;
2. Koenig S H, Brown R D III. Relaxometry of Tissue. In: Gupta R K, editor. NMR spectroscopy of cells and organisms, vol. II. Boca Raton, Fla.: CRC Press; 1987. p 75-114;
3. Koenig S H, Brown R D III. Field-cycling relaxometry of protein solutions and tissue: implications for MRI. Progr NMR Spectrosc 1990; 22: 487-567;
4. Noack F. NMR field-cycling spectroscopy: principles and applications. Progr NMR Spectrosc 1986; 18:171-276;
5. Lee S K, Mossle M, Myers W, Kelso N, Trabesinger A H, Pines A, Clarke J. SQUID-detected MRI at 132 microT with T1-weighted contrast established at 10 microT-300 mT. Magn Reson Med. 2005 January; 53(1):9-14;
6. Lu H, Golay X, Pekar J J, Van Zijl P C. Functional magnetic resonance imaging based on changes in vascular space occupancy. Magn Reson Med. 2003 August; 50(2):263-74;
7. W-C Wu, S Wegener, R B Buxton, E C Wong. Vascular space occupancy weighted imaging with control of inflow effect and higher signal-to-noise ratio. Proc. Intl. Meeting ISMRM, p. 2768, 2006;
8. Venook R D, Matter N I, Ramachandran M, Ungersma S E, Gold G E, Giori N J, Macovski A, Scott G C, Conolly S M. Prepolarized magnetic resonance imaging around metal orthopedic implants. Magn Reson Med. 2006 July; 56(1):177-86;
9. Ungersma S E, Matter N I, Hardy J W, Venook R D, Macovski A, Conolly S M, Scott G C. Magnetic resonance imaging with T1 dispersion contrast. Magn Reson Med. 2006 June; 55(6):1362-71;
10. Matter N I, Scott G C, Grafendorfer T, Macovski A, Conolly S M. Rapid polarizing field cycling in magnetic resonance imaging. IEEE Trans Med Imaging. 2006 January; 25(1):84-93;
11. Morgan P, Conolly S, Scott G, Macovski A. A readout magnet for prepolarized MRI. Magn Reson Med. 1996 October; 36(4):527-36;
12. Zhao F, Wang P, Hendrich K, Kim S G. Spatial specificity of cerebral blood volume-weighted fMRI responses at columnar resolution. Neuroimage 2005. 15;27(2): 416-24;
13. P C M Van Zijl, H Lu, X Golay. Microvascular blood volume magnetic resonance imaging. US Patent Application 20050215881. Sep. 29, 2005;
14. X Golay, H Lu, P C M van Zijl. Methods for multiple acquisitions with global inversion cycling for vascular-space-occupancy dependant and apparatuses and devices related thereto. U.S. Pat. No. 7,071,689. Jul. 4, 2006;
15. G. McKinnon. Magnetic resonance imaging apparatus. U.S. Pat. No. 6,462,544. Oct. 8, 2002;
16. European Union grant: Seventh Framework Programme FP7/2007-2013, HEALTH-F5-2008-200859;
17. V S Zotev, P L Volegov, A N Matlashov, M A Espy, J C Mosher, R H Kraus. Parallel MRI at microtesla fields. J. Magn. Reson. 2008. 192(2): 197-208;
18. V S Zotev, A N Matlashov, P L Volegov, I M Savukov, M A Espy, J C Mosher, J J Gomez, R H Kraus. Microtesla MRI of the human brain combined with MEG. J. Magn. Reson. 2008. 194: 115-120.

What is claimed is:

1. A method comprising:
(a) generating a bulk magnetization in a subject;
(b) establishing a longitudinal relaxation time constant (T₁) contrast between constituents in the subject;
(c) creating spatially localized magnetic resonance (MR) signals from the subject;
(d) detecting the MR signals; and
(e) generating a functional image of the subject.

2. The method of claim 1, wherein operation (a) comprises applying at least one initial polarizing magnetic field (Bₚ) pulse to the subject.

3. The method of claim 1, wherein operation (b) comprises applying a magnetic field-cycled pulse sequence to the subject.

4. The method of claim 3, wherein magnetic field-cycled pulse sequence includes at least one pulse selected from the group consisting of a constant evolution magnetic field pulse (Bₑ), an oscillating magnetic field pulse (B₁), and an additional polarizing magnetic field pulse (Bₚ).

5. The method of claim 1, wherein the subject includes brain tissue.

6. The method of claim 5, wherein operation (b) comprises placing a blood magnetization of the brain tissue at a null value at the time of operation (d).

7. The method of claim 5, wherein operation (b) comprises placing a blood magnetization of the brain tissue near a null value at the time of operation (d).

8. The method of claim 1, wherein operation (c) comprises applying at least one readout oscillating and gradient magnetic field pulse sequence to the subject.

9. The method of claim 1, wherein operation (d) comprises detecting the MR signals via magnetic induction.

10. The method of claim 9, wherein operation (d) is performed via magnetic induction with at least one inductive pickup coil.

11. The method of claim 9, wherein operation (d) is performed via magnetic induction with at least one magnetometer.

12. The method of claim 9, wherein operation (d) is performed via magnetic induction with a device selected from the group consisting of at least one superconducting quantum interference device (SQUID) and a plurality of SQUIDS.

13. The method of claim 12, further comprising constraining the localization of magnetoencephalography (MEG) signals with the detected MR signals.

14. The method of claim 1, wherein operation (e) comprises measuring functional activation in the subject from the detected MR signals.

15. The method of claim 14, wherein operation (e) further comprises outputting a representation of the measured functional activation.

16. The method of claim 1, wherein operation (e) comprises measuring functional deactivation in the subject from the detected MR signals.

17. The method of claim 16, wherein operation (e) further comprises outputting a representation of the measured functional deactivation.

18. An apparatus comprising:
a pre-polarizing magnet;
a secondary magnet;
gradient electromagnetic coils;
rotating magnetic field transmission coils;
a detection mechanism selected from the group consisting of one or more magnetometers and one or more inductive pickup coils; and
a controller comprising program instructions for conducting a process comprising the operations of:
(a) generating a bulk magnetization in a subject using the pre-polarizing magnet;
(b) establishing a longitudinal relaxation time constant (T₁) contrast between constituents in the subject using a sequence of constant and time-varying magnetic fields;
(c) creating spatially localized magnetic resonance (MR) signals from the subject using an imaging pulse sequence;
(d) detecting the MR signals using the detection mechanism; and
(e) generating a functional image of the subject.
